(19) United States
(12) Patent Application Publication
(10) Pub. No.: US 2020/0372636 A1
(43) Pub. Date: Nov. 26, 2020

(54) SYSTEM METHOD AND COMPUTER-ACCESSIBLE MEDIUM FOR DETERMINING BREAST CANCER RESPONSE USING A CONVOLUTIONAL NEURAL NETWORK

(71) Applicant: THE TRUSTEES OF COLUMBIA UNIVERSITY IN THE CITY OF NEW YORK, NEW YORK, NY (US)

(72) Inventor: RICHARD HA, White Plains, NY (US)

(21) Appl. No.: 16/766,265

(22) PCT Filed: Nov. 21, 2018
(86) PCT No.: PCT/US2018/062319

§ 371 Date: May 21, 2020

Related U.S. Application Data
(60) Provisional application No. 62/589,924, filed on Nov. 22, 2017.

(51) Int. Cl. G06T 7/00; G16H 50/30; G06T 5/50; G06T 3/40; G06T 3/60; G06N 3/04

(52) U.S. Cl. CPC G06T 7/0012; G16H 50/30; G06T 5/50; G06T 3/40; G06T 3/60; G06N 3/0454; G06T 2207/20132; G06T 2207/30096; G06T 2207/20084; G06T 2207/10096; G06T 2207/20081; G06T 2207/20224; G06T 2207/30068

(57) ABSTRACT

An exemplary system, method and computer-accessible medium for determining a breast cancer response(s) for a patient(s) can include, for example, receiving an image(s) of an internal portion(s) of a breast of the patient(s), and determining the breast cancer response(s) by applying a neural network(s) to the image(s). The breast cancer response(s) can be a response to at least one chemotherapy treatment. The breast cancer response(s) can include an Oncotype DX recurrence score. The breast cancer response(s) can be a neoadjuvant axillary response. The image(s) can be a magnetic resonance image(s) (MRI). The MRI(s) can include a dynamic contrast enhanced MRI(s).

### CROSS-REFERENCE TO RELATED APPLICATION(S)

This application relates to and claims priority from U.S. Patent Application No. 62/589,924, filed on Nov. 22, 2017, the entire disclosure of which is incorporated herein by reference.

### FIELD OF THE DISCLOSURE

The present disclosure relates generally to determining information regarding breasts and breast tissue, and more specifically, to exemplary embodiments of exemplary systems, methods and computer-accessible medium for determining breast cancer response using a convolutional neural network.

### BACKGROUND INFORMATION

Breast cancer is one of the most ubiquitous malignancies afflicting women worldwide, and is the second most common cause of cancer deaths among women in the United States. (See, e.g., Reference 37). Not all breast cancers are the same, with a wide spectrum of intrinsic biologic diversity seen across multiple subtypes indicating variable biologic behavior and treatment options. (See, e.g., Reference 38). If a patient meets the criteria of estrogen receptor positive (“ER+”), human epidermal growth factor receptor-2 negative (“HER2−”), and node-negative, adjuvant chemotherapy may not be indicated, as the risk of recurrence is comparable to the harm from toxicity. (See, e.g., References 39 and 40). These patients can receive surgery, endocrine therapy, or radiation. (See, e.g., Reference 40).

Oncotype Dx (Genomic Health, Redwood City, Calif.) is a validated 21-gene reverse transcriptase polymerase chain reaction (“RT-PCR”) assay involved in tumor cell proliferation and hormonal response, which provides a recurrence score (“RS”) to quantitatively predict outcomes in patients who meet the criteria of ER+/HER2−/node negative invasive breast carcinoma. (See, e.g., References 41-43). In 2016 the updated guidelines of the American Society of Clinical Oncology (“ASCO”) recommended use of this RS in ER+/HER2−/node negative breast cancer to help determine the utility of adjuvant systemic chemotherapy. (See, e.g., Reference 39).

Although effective, genetic analysis such as Oncotype Dx is invasive and expensive, which has motivated the investigation of imaging analysis to determine tumor heterogeneity. Magnetic resonance imaging (“MRI”) is a common modality used in the diagnosis of breast cancer given its high soft-tissue contrast and sensitivity. (See, e.g., Reference 44). In recent years there have been investigations into quantitative analysis of specific extracted imaging features termed “radiomics.” Further correlation of these quantitative imaging features to molecular gene expression defines “radiogenomics.” (See, e.g., Reference 45).

The field of radiomics and radiogenomics has developed largely due to the contribution of machine-learning procedures utilizing the extraction of pertinent imaging features and correlating with clinical data. More recently, due to advances in the computer hardware technology, a subset of machine learning utilizing a type of artificial neural network called convolutional neural networks (“CNNs”) has begun to proliferate for medical imaging analysis. In contrast to traditional procedures that utilize hand-crafted features based on human extracted patterns, neural networks facilitate the computer to automatically construct predictive statistical models, tailored to solve a specific problem subset. (See, e.g., Reference 46). The laborious task of human engineers inputting specific patterns to be recognized could be replaced by inputting curated data and facilitating the technology to self-optimize and discriminate through increasingly complex layers.

Neoadjuvant chemotherapy (“NAC”) has become a widely used treatment approach in the management of breast cancer. In addition to the established benefits of increasing rates of operability and breast-conservation for locally-advanced tumors, NAC facilitates the assessment of the clinical efficacy of novel systemic combinations and targeted therapies in vivo within a treatment-naïve patient population. (See, e.g., Reference 1).

Several large randomized neoadjuvant trials have demonstrated pathological complete response (“pCR”) to be a potential surrogate marker for clinical efficacy as there can be a significant correlation between patients who achieved a pCR and improved disease-free and overall survival. (See, e.g., References 2 and 3). This association varies among subtypes, with the strongest diagnostic accuracy seen in HER2 positive and triple-negative breast cancer. (See, e.g., References 4 and 5). While systemic treatments delivered in the adjuvant setting needs many years of follow-up to validate a clinical benefit, pCR serves as an attractive surrogate end point for improved long-term outcome after only several weeks of neoadjuvant therapy. (See, e.g., References 6 and 7).

Axillary lymph node pCR has been shown to be a dominant prognostic factor in long-term outcome across all breast cancer subtypes. A large prospective study, including 403 patients with proven axillary lymph node metastases who underwent NAC followed by sentinel lymph node biopsy (“SLND”) or ALND showed 22% achieved axillary pCR, of which 69% achieved pCR of the primary tumor. The overall survival (“OS”) in patients who achieved axillary pCR was significantly higher compared with those with axillary residual disease (93% [95% confidence interval [CI] 87.5-98.5] vs. 72% [95% CI 66.5-77.5], P<0.0001). In patients who achieved axillary pCR, there was no significant difference in recurrence-free survival (“RFS”) or OS in those who had residual primary disease versus achieved primary tumor pCR. Although limited by a small sample size, these findings suggest residual primary tumor in the setting of axillary pCR does not infer a worse prognosis, possibly secondary to a difference in metastatic potential of the tissues of the axilla compared to the breast. (See, e.g., Reference 69). The prognostic value of axillary lymph node status accurately assesses the treatment response critical in the management of breast cancer.

Advances in genomics have demonstrated breast cancer to be a disease with a spectrum of biologically relevant molecular subtypes. This significant disease heterogeneity poses a major challenge in the development of novel treatments. Targeted therapies may only be effective in a small subset of breast cancers, which has contributed to the difficulty establishing a therapeutic benefit in a large, heterogeneous, clinical trial. (See, e.g., References 8 and 9). There can be potential for significant clinical benefit in streamlining the testing of novel NAC with early response assessment and prediction. This can be the goal of the ongoing adaptive neoadjuvant I-SPY 2 (Investigation of Serial Studies to Predict Your Therapeutic Response with Imaging and Molecular Analysis 2) trials, which have already “graduated” neratinib in HER2 positive disease and veliparib-carboplatin in triple-negative disease. (See, e.g., References 10 and 11). Timely identification of responders to therapy can reduce the time, cost, and patient numbers needed to identify new beneficial therapies. Furthermore, early identification of non-responders can be beneficial in minimizing the potential toxicity of ineffective treatments and the delay of further exploration into potential alternative preoperative therapy. (See, e.g., Reference 12).

Quantitative MRI has emerged as a powerful imaging modality in the neoadjuvant treatment response assessment and identification of potential imaging-based biomarkers, with successful incorporation into the clinical trial setting. (See, e.g., References 13-16). Recent approaches have correlated changes in specific morphologic and kinetic parameters between a baseline and interval MRI after the initiation of chemotherapy, as early as after the first cycle, to predict treatment response and pCR. Further integration of clinically-relevant mathematical models to account for biologic features of tumor growth and treatment response have enhanced the predictive accuracy of these methods. (See, e.g., References 17 and 18). The vast majority of current models in early-response assessment depend on interval imaging after the initiation of therapy, without the ability to successfully determine a priori treatment response or pCR, prior to the initiation of treatment given the challenges of tumor heterogeneity. (See, e.g., References 13-18).

Deep learning through CNNs has demonstrated strong performance in various image classification tasks in recent years with a growing number of applications. (See, e.g., Reference 19). Deep learning methods facilitate a machine to extract high-level information from raw input images using several non-linear modules to amplify important features for image discrimination and classification. Machine learning can be further supervised using adjustable parameters to intricately correlate specific inputs and outputs.

While radiographic complete response (“rCR”) of the primary tumor can be determined objectively by the lack of residual enhancement, axillary rCR can be challenging given variability of normal lymph node morphology and enhancement pattern. MRI before and after NAC in correlation with pathologic evaluation was examined in 128 patients with breast cancer and demonstrated axillary rCR to only achieve a negative predictive value (“NPV”) of 66.7% and a positive predictive value (“PPV”) of 65.6%. (See, e.g., Reference 71). An additional prospective examination correlating MRI before and after NAC with axillary biopsy results in 43 patients with breast cancer showed that pre-NAC MRI was significantly associated with pathology (P=0.014) with a false-positive rate, false-negative rate, sensitivity, and specificity of 50, 3, 97, and 50%, respectively. However, post-NAC MRI was not predictive of surgical pathologic findings (P=0.342), with a false-positive rate, false-negative rate, sensitivity, and specificity of 38, 46, 55, and 63%, respectively. (See, e.g., Reference 79). The results of these studies demonstrate the challenges of accurately assessing the axilla in the post-NAC setting.

Management of breast cancer, specifically in axillary metastasis, has shown a continuous trend toward less invasive therapy, with initially accepted ALND largely replaced by SLND. The National Surgical Adjuvant Breast and Bowel Project (“NSABP”) B-32 trial showed sentinel lymph node identification to have a success rate of 96.2% with a false-negative rate of 6.7%. (See, e.g., Reference 80). However, initially, there was a concern for an accurate pathologic analysis of SLND after NAC. A large meta-analysis, including 10 studies with a total of 449 patients with clinically node-negative disease who underwent SLND after NAC, showed a pooled identification rate of 94.3% with a false-negative rate (“FNR”) of 7.4%, which can be comparable to the standard accepted identification rates of 88-97% and FNRs of 5-12%. (See, e.g., Reference 81).

However, SLND after NAC in node-positive breast cancer remains a point of controversy. Fibrosis of the axilla after chemotherapy alters lymphatic drainage and increases difficulty of surgical dissection. A large, prospective study that included 525 patients with cN1 disease who underwent SLND followed by ALND showed approximately 41% achieved pCR with a FNR as high as 12.6%. Although the FNR was higher than the pre-specified threshold of 10%, the rate was lowered with application of dual-agent mapping procedure and evaluation of three or more lymph nodes. (See, e.g., Reference 78). A better evaluation of an axillary treatment response using noninvasive imaging procedures has a potential to significantly impact breast cancer patients, particularly in node-positive disease.

Thus, it may be beneficial to provide an exemplary system method and computer-accessible medium for determining breast cancer response using a convolutional neural network which can overcome at least some of the deficiencies described herein above.

### SUMMARY OF EXEMPLARY EMBODIMENTS

An exemplary system, method and computer-accessible medium for determining a breast cancer response(s) for a patient(s) can include, for example, receiving an image(s) of an internal portion(s) of a breast of the patient(s), and determining the breast cancer response(s) by applying a neural network(s) to the image(s). The breast cancer response(s) can be a response to at least one chemotherapy treatment. The breast cancer response(s) can include an Oncotype DX recurrence score. The breast cancer response(s) can be a neoadjuvant axillary response. The image(s) can be a magnetic resonance image(s) (MRI). The MRI(s) can include a dynamic contrast enhanced MRI(s).

In some exemplary embodiments of the present disclosure, the neural network can include a convolutional neural network (CNN). The CNN can include a plurality of layers. The layers can include (i) a plurality of combined convolutional and rectified linear unit (ReLu) layers, (ii) a plurality of max pooling layers, (iii) a combined fully connected and ReLu layer(s), and (iv) a dropout layer(s). The combined convolutional and rectified linear unit (ReLu) layers can include at least ten combined convolutional and rectified linear unit (ReLu) layers, and the max pooling layers can include at least four max pooling layers. Two of the at least ten combined convolutional and rectified linear unit (ReLu) layers can have 64×64×64 feature channels, two of the at least ten combined convolutional and rectified linear unit (ReLu) layers can have 32×32×128 feature channels, three of the at least ten combined convolutional and rectified linear unit (ReLu) layers can have 16×16×128 feature channels, and three of the at least ten combined convolutional and rectified linear unit (ReLu) layers can have 8×8×512 feature channels.

In certain exemplary embodiments of the present disclosure, a score(s) can be determined based on the image(s) using the neural network(s). The breast cancer response(s) can be determined based on the score. The breast cancer response(s) can be determined based on the score being above 0.5. The image can be normalized by, for example, subtracting a mean for a plurality of images of further internal portions of further breasts, and dividing by a standard deviation for the image(s). The image(s) can be translated, rotated, scaled, and sheared.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIGS. 1A-1C are exemplary T1 post contrast breast MRI images of tumors with complete pathologic response according to an exemplary embodiment of the present disclosure;

FIGS. 2A-2C are exemplary T1 post contrast breast MRI images of tumors with partial pathologic response according to an exemplary embodiment of the present disclosure;

FIGS. 3A-3C are exemplary T1 post contrast breast MRI images of tumors with no pathologic response according to an exemplary embodiment of the present disclosure;

FIG. 4 is an exemplary schematic diagram of an exemplary convolutional neural network according to an exemplary embodiment of the present disclosure;

FIG. 5 is an exemplary graph illustrating receiver operating characteristics for a three-class CNN prediction of NAC treatment response according to an exemplary embodiment of the present disclosure;

FIG. 6 is an exemplary diagram of image pre-processing according to an exemplary embodiment of the present disclosure;

FIG. 7A is an exemplary set of DCE tumor images corresponding to a low Oncotype DX recurrence score according to an exemplary embodiment of the present disclosure;

FIG. 7B is an exemplary set of DCE tumor images corresponding to an intermediate Oncotype DX recurrence score according to an exemplary embodiment of the present disclosure;

FIG. 7C is an exemplary set of DCE tumor images corresponding to a high Oncotype DX recurrence score according to an exemplary embodiment of the present disclosure;

FIG. 8 is an exemplary schematic diagram of a further exemplary convolutional neural network according to an exemplary embodiment of the present disclosure;

FIG. 9 is an exemplary graph illustrating receiver operating characteristics for a three-class CNN prediction procedure according to an exemplary embodiment of the present disclosure;

FIG. 10 is an exemplary graph illustrating receiver operating characteristics for a two-class CNN prediction procedure according to an exemplary embodiment of the present disclosure;

FIGS. 11A-11C are exemplary T1 post-contrast breast MRI images of tumors from patient with pCR of the axilla according to an exemplary embodiment of the present disclosure;

FIGS. 12A-12C are exemplary T1 post-contrast breast MRI images of tumors from patient with non-pCR of the axilla according to an exemplary embodiment of the present disclosure;

FIG. 13 is an exemplary graph illustrating receiver operating characteristics for a two class CNN prediction of NAC treatment response of the axilla according to an exemplary embodiment of the present disclosure;

FIG. 14 is an exemplary flow diagram of a method for determining breast cancer response for a patient according to an exemplary embodiment of the present disclosure; and

FIG. 15 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

The exemplary system, method, and computer-accessible medium, according to an exemplary embodiment of the present disclosure, can include an exemplary determination breast cancer response using various exemplary imaging modalities. For example, the exemplary system, method, and computer-accessible medium according to an exemplary embodiment of the present disclosure is described herein using mammographic images and/or optical coherence tomography (“OCT”) images. However, the exemplary system, method, and computer-accessible medium according to an exemplary embodiment of the present disclosure can also be used on other suitable imaging modalities, including, but not limited to, magnetic resonance imaging, positron emission tomography, ultrasound, and computed tomography.

### Exemplary Prediction Breast Tumor Response to Chemotherapy

### Exemplary Patient Selection and Eligibility

A retrospective review of identified 141 patients with the diagnosis of breast cancer between Jan. 1, 2005 and Jun. 1, 2016. All patients met the following criteria: (i) underwent a staging breast MRI prior to the initiation of therapy; (ii) received adriamycin-based and/or taxane-based neoadjuvant chemotherapy with additional HER2 directed therapy (e.g., trastuzumab/pertuzumab) in patients with HER2 positive tumor; and (iii) successfully underwent surgical resection of their primary breast tumor with appropriate lymph node sampling.

### Exemplary Pathologic Analysis

Data on tumor pathologic characteristics were obtained from the original pathology reports of the core biopsy specimen. Breast tumor subtype was determined based on immunohistochemical (“IHC”) staining of the ER and progesterone receptor (“PR”) interpreted according to the American Society of Clinical Oncology and College of American Pathologists Guidelines. Tumors were considered receptor positive if either ER or PR demonstrated greater than about 1% positive staining. (See, e.g., Reference 21). Tumors were considered HER2 positive if they were 3+ by immunohistochemistry or demonstrated gene amplification with a ratio of HER2/CEP17>2 by in situ hybridization. (See, e.g., Reference 22). Breast tumor subtypes were defined as follows: (i) Luminal A (e.g., ER/PR positive, HER2 negative); (ii) luminal B (e.g., ER/PR positive, HER2 positive); (iii) HER2 positive (e.g., ER/PR negative, HER2 positive); and (iv) triple negative or basal-like (e.g., ER/PR and HER2 negative). Clinical and pathologic staging was determined based on the American Joint Committee on Cancer TNM Staging Manual, 7th edition. Patients were classified into 3 groups based on their NAC response confirmed on final surgical pathology: Pathologic complete response (group 1), partial response (group 2) and no response/progression (group 3). pCR was defined as no residual invasive disease in the breast or lymph nodes on surgical pathology specimens (ypTO/Tis ypNO).

### Exemplary MRI Methods

An exemplary MRI procedure was performed on a 1.5-T or 3.0-T commercially available system using an eight-channel breast array coil. A bilateral sagittal T1-weighted fat-suppressed fast spoiled gradient-echo sequence (17/2.4; flip angle, 35°; bandwidth, 31-25 Hz) was then performed before and after a rapid bolus injection (gadobenate dimeglumine/Multihance; Bracco Imaging; 0.1 mmol/kg) delivered through an IV catheter. Image acquisition started after contrast material injection, and was obtained consecutively with each acquisition time of 120 seconds. Section thickness was 2-3 mm using a matrix of 256×192 and a field of view of 18-22 cm. Frequency was in the antero-posterior direction.

### Exemplary Computer-Based Image Analysis: Image Preprocessing

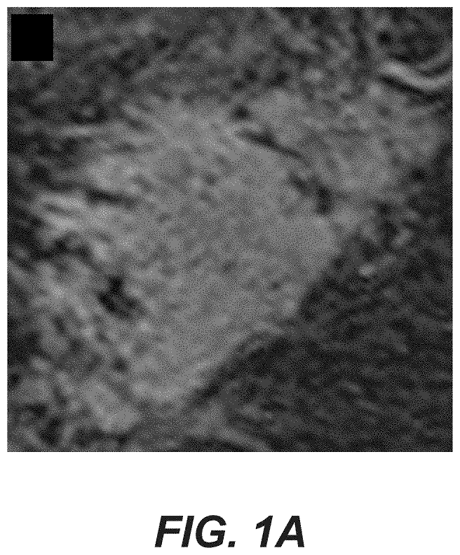

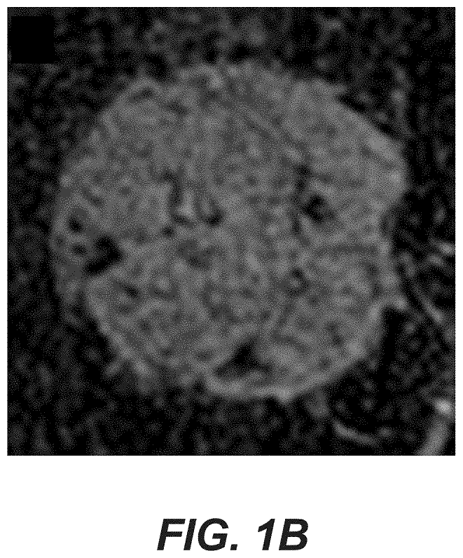

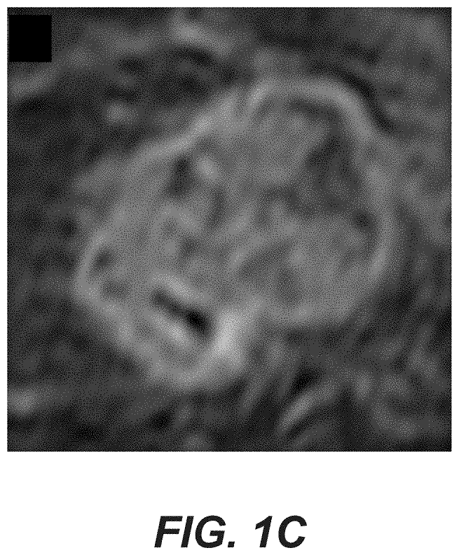

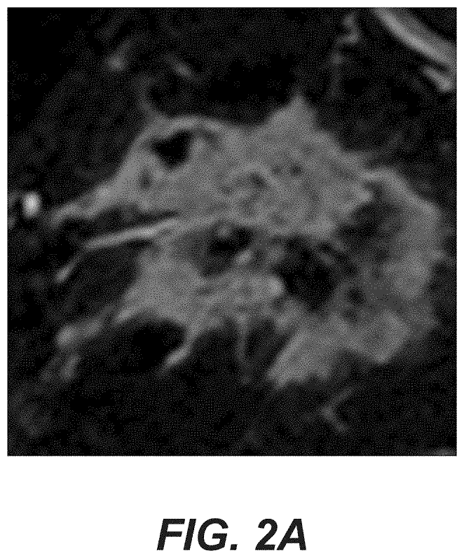

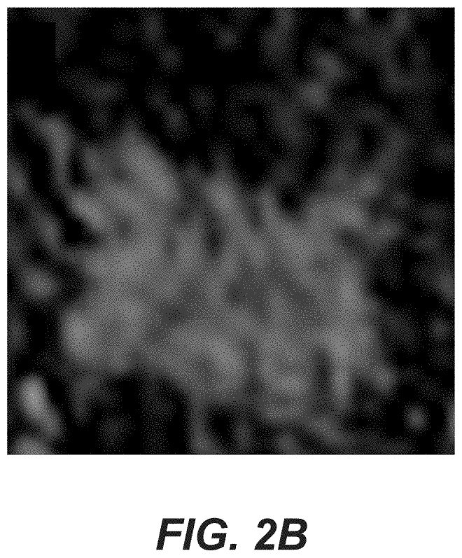

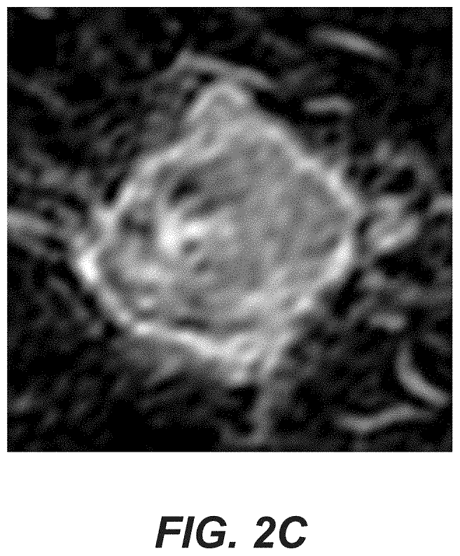

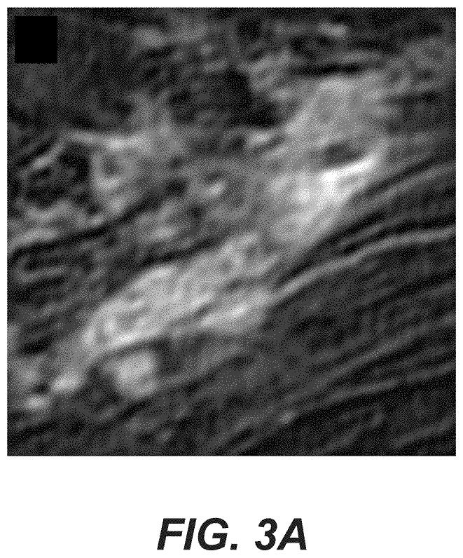

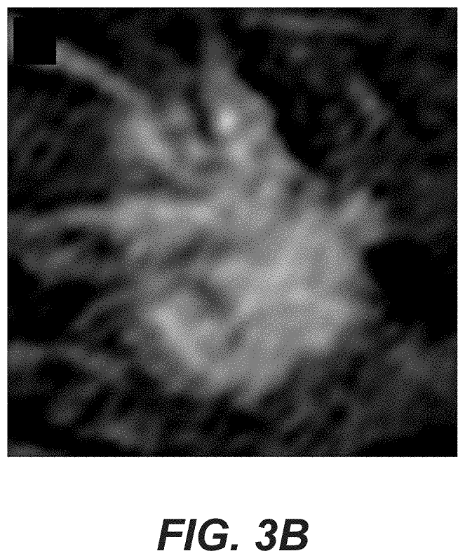

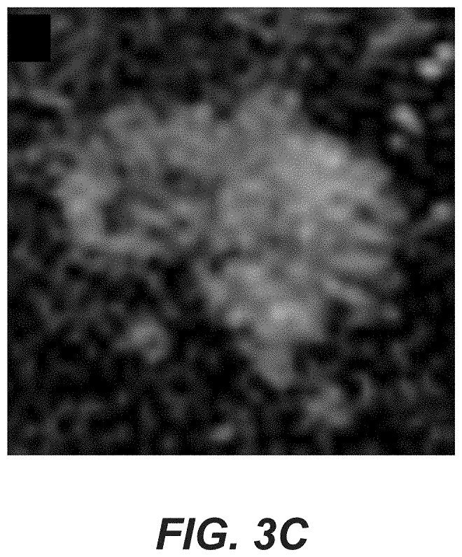

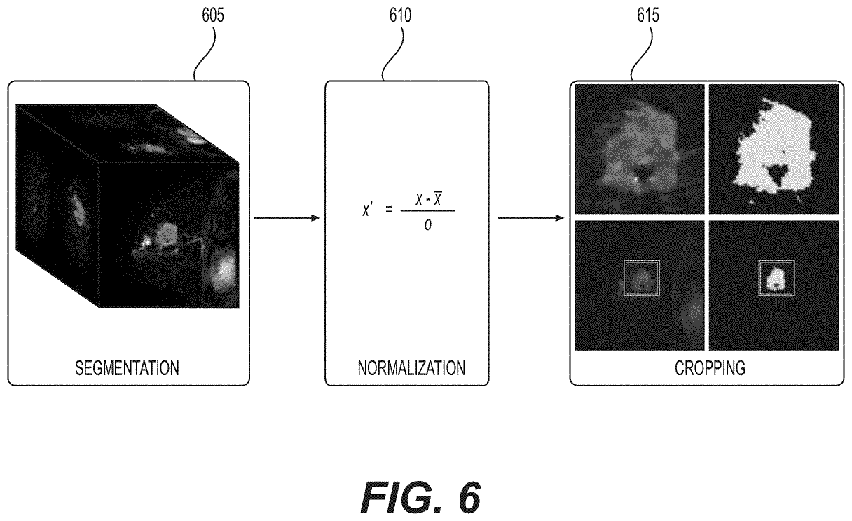

As shown in the images of FIGS. 1A-1C, 2A-2C and 3A-3C, for each breast MRI, a tumor was identified on first T1 post contrast dynamic images. As illustrated in the diagram of FIG. 6, the entire breast volume underwent 3D segmentation 605 by a breast fellowship trained radiologist with 8 years of experience using an open source software platform 3D Slicer. (See, e.g., Reference 23). A total of 3107 volumetric slices for 141 tumors were collected. The data was normalized 610 by subtracting the mean intensity value of each slice and by dividing by the standard deviation of each slice . . . . A 64×64 voxel crop 615 of the segmented tumor was then input into the exemplary CNN. An average of 22 slices of volumetric data per tumor was used, with a threshold of 75 voxels per slice. At the time of training, real time data augmentation was performed to limit over-fitting of data. Using an exemplary random affine transformation, additional images were created by modifying the images including, (i) randomly rotating images (e.g., range 10 degrees), (ii) horizontally flipping images, (iii) shearing images (e.g., range 0.1), and (iv) zooming in one images (e.g., range 0.1).

### Exemplary CNN Architecture

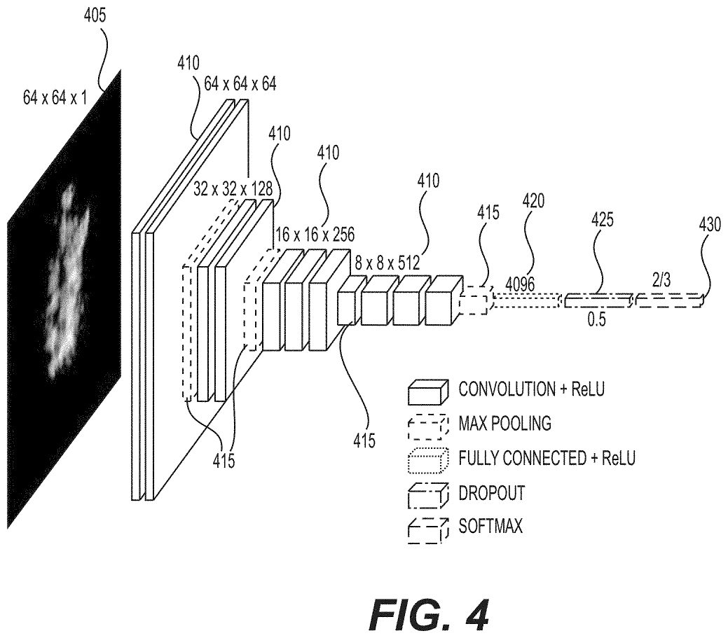

FIG. 4 shows a diagram of an exemplary CNN according to an exemplary embodiment of the present disclosure. An exemplary block consists of multiple convolution layers of 3×3 convolution kernels that have progressively increasing feature channels in deeper layers. The convolution layers can be followed by the nonlinear rectified linear unit activation function (“ReLu”). (See, e.g., Reference 25). Before each increase of feature channels, a 2×2 max pooling layer can be applied to reduce the amount of parameters and computation in the network, serving the double purpose of controlling overfitting. Four of these blocks can be stacked on each other before the architecture flattens out to a full connected dense layer. The fully connected layer acts as a perceptron and can be mathematically similar to a least squares regression. Dropout of 25% can be applied in the dense layer to prevent overfitting by limiting co-adaptation of parameters. (See, e.g., Reference 24). L2 regularization with a beta of 0.01 can be used after the dense layer to place a penalty on the squared magnitude of the kernel weights. This penalizes outlier parameters and in encourages generalizable parameters. This reduces overfitting in the model and leads to a more generalizable model. A softmax classifier can be used for the loss function.

As shown in the exemplary diagram of FIG. 4, an input 405 can be provided into a plurality of combined convolution and ReLu layers 410. Multiple max pooling layers 415 can be interspersed within the combined convolution and ReLu layers 410. The combined convolution and ReLu layers can feed into a combined fully connected convolution and ReLu layer 420. A dropout layer 425 can provide an output to softmax 430 in order to determine a chemotherapy response.

### Exemplary CNN Training

The exemplary data was divided into a validation set, which included 80% of the data, and a test set, which included 20% of the data. The validation test set was then divided into 5 folds, and 5 fold cross validation was performed. Training from scratch without pretrained weights was performed over 100 epochs using adam optimizer with nesterov momentum at an initial learning rate of 0.002. Each of the 5 models was tested against the 20% hold out data to obtain sensitivity, specificity and accuracy. Receiver operator curves were also calculated for each of the 5 models.

### Exemplary Patient Tumor Pathology and Response

A total of 141 patients met the criteria for inclusion in this study. Three class neo-adjuvant prediction model was evaluated for the three patient groups. The breakdown of tumor response and molecular subtype is shown in Table 1 below. Group 1 included 46 patients with pathologic complete response. Group 2 included 57 patients with partial response. Group 3 included 38 patients with no response to progression on chemotherapy. The molecular subtype based on IHC staining included: (i) 61 luminal A; (ii) 39 luminal B; (iii) 16 HER2 positive; and (iv) 25 triple negative or basal-like.

TABLE 1
Pathologic tumor response and molecular subtype

| | |
|---|---|
| Molecular | Pathologic Response |

| Subtype | Complete | Partial | No/progression | Total |
|---|---|---|---|---|
| Luminal A | 11 | 24 | 26 | 61 |
| Luminal B | 18 | 17 | 4 | 39 |
| HER2+ | 8 | 6 | 2 | 16 |
| Triple− | 9 | 10 | 6 | 25 |
| Total | 46 | 57 | 38 | 141 |

The rate of pCR is shown in Table 2 below, demonstrating: (i) 18% (11/61) of the luminal A, (ii) 46% (18/39) of the luminal B group, (iii) 50% (8/16) of the HER2 positive group, (iv) and 36% (9/25) of the triple negative group achieved pCR. Combined luminal B, HER2 positive, and triple-negative tumors had a significantly higher rate of pCR compared to luminal A, with a rate of 44% (35/80) versus 18% (11/61) respectively (p=0.002).

TABLE 2
Rate of pCR per molecular subtype

| | |
|---|---|
| Molecular | Pathologic Response |
| Subtype | Complete |

| | | |
|---|---|---|
| Luminal A | 11/61 | 18% |
| Luminal B | 18/39 | 46% |
| HER2+ | 8/16 | 50% |
| Triple− | 9/25 | 36% |

The rate of no response/progression of disease is shown in Table 3 below, demonstrating: (i) 43% (26/61) of the luminal A group, (ii) 10% (4/39) of the luminal B group, (iii) 13% (2/16) of the HER2 positive group, and (iv) 24% (6/25) of the triple negative group showed no treatment response or progression of disease. Luminal A tumors had a significantly higher rate of no response/progression compared to the other three groups, with a rate of 43% (26/61) versus 15% (12/80) respectively (p=0.0005).

TABLE 3
Rate of no response/progression per molecular subtype

| | |
|---|---|
| Molecular | Pathologic Response |
| Subtype | No/Progression |

| | | |
|---|---|---|
| Luminal A | 26/61 | 43% |
| Luminal B | 4/39 | 10% |
| HER2+ | 2/16 | 13% |
| Triple− | 6/25 | 24% |

### Exemplary CNN Statistical Analysis

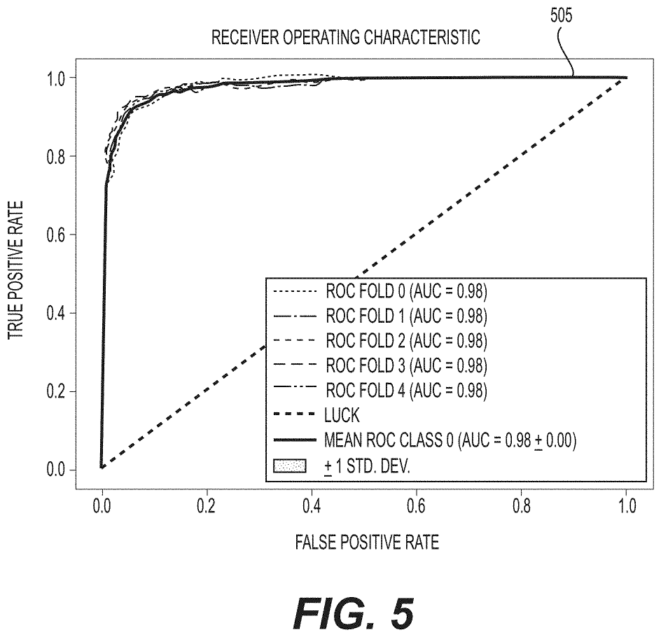

The confusion matrix, shown in table 4 below, shows the exemplary CNN predicted class of the hold out test data versus the true class of the hold out test data. The values represent the average number of slices over the five folds of cross validation plus or minus the standard deviation. A final softmax score threshold of 0.5 was used for classification. The exemplary CNN achieved an overall mean accuracy of 88% (95% CI, f 0.6%) in three class prediction of NAC treatment response on a five-fold validation accuracy test. FIG. 5 shows an exemplary graph of an ROC plot (e.g., mean ROC 505) according to an exemplary embodiment of the present disclosure. Three class prediction discriminating one class from the other two was analyzed. Group 1 (complete response) had a specificity of 95.1% f 3.1%, sensitivity of 73.9% f 4.5%, and accuracy of 87.7% f 0.6%. Group 2 (partial response) had a specificity of 91.6% f 1.3%, sensitivity of 82.4% f 2.7%, and accuracy of 87.7% f 0.6%. Group 3 (no response/progression) had a specificity of 93.4% f 2.9%, sensitivity of 76.8% f 5.7%, and accuracy of 87.8% f 0.6%.

TABLE 4
Convolution Neural Network Performance Confusion Matrix

| | Predicted Response | | |
|---|---|---|---|
| True | | | No |
| Response | Complete | Partial | Response |
| Complete | 160.2 ± 4.4 | 10.6 ± 2.9 | 8.6 ± 2.9 |
| Partial | 9.2 ± 4.2 | 219.6 ± 5.7 | 18.2 ± 5.4 |
| No | 10.8 ± 4.3 | 19.2 ± 3.1 | 165 ± 6.7 |
| Response | | | |

Prior to initiation of therapy, the exemplary CNN procedure achieved an overall accuracy of 88% in predicting NAC response in patients with locally advanced breast cancer. The exemplary results demonstrate that the exemplary system, method, and computer-accessible medium can utilize a CNN to predict NAC response prior to initiation of therapy. This represents an improved approach to early treatment response assessment based on a baseline breast MRI obtained prior to the initiation of treatment, and significantly improves on current prediction methods that rely on interval imaging after the initiation of therapy.

Although there has been significant progress in MRI to assess therapy response, the vast majority of studies thus far depend on interval imaging after initiation of therapy. Quantitative imaging procedures have become an active area of research given the limitations of qualitative tumor response assessment using the Response Evaluation Criteria in Solid Tumors (“RECIST”). (See, e.g., Reference 27). Quantitative methods of response assessment have examined changes in kinetic parameters (e.g., volume transfer constant Ktrans, exchange rate constant kept) in dynamic contrast-enhanced MRI (“DCE-MRI”), (see, e.g., References 28-30) as well as morphologic changes (e.g., three-dimensional volume, signal enhancement ratio, tissue cellularity) using DCE-MRI, and diffusion-weighted MRI (“DW-MRI”) with predictive value after one or more cycles of therapy. (See, e.g., References 14, 15 and 31). The limitations of these methods include the often delayed morphologic-based changes that occur despite treatment-induced biologic response that may not be reflected by imaging performed during or shortly after completion of therapy. By incorporating a mechanically coupled reaction-diffusion model using patient-specific imaging data to drive a biomechanical model of tumor growth, improved prediction of therapy response as compared to prior procedures can be achieved, resulting in a sensitivity and specificity of 92% and 84%, respectively. (See, e.g., Reference 32). While significant advances in response assessment have been shown, the previously described studies all rely on interval imaging after initiation of therapy.

Currently available clinical and pathologic data shows luminal B, HER2-positive, and triple-negative breast cancer responds best to NAC. A large meta-analysis of thirty studies including 11,695 patients investigating pCR after NAC showed average rates of pCR were 8.3% in luminal A, 18.7% in luminal B, 38.9% in HER2 positive, and 31.1% in triple-negative breast cancer subtypes. (See, e.g., Reference 33). Similarly, HER2-positive, and triple-negative tumors achieved significantly higher pCR, compared to the luminal A subtype using the exemplary CNN. While this information can be helpful, it cannot be used solely to predict who can respond to NAC given over half of these patients do not have pCR.

### Exemplary Prediction of Oncotype Dx Recurrence Score

### Exemplary MRI Acquisition and Analysis

An exemplary MRI procedure was performed on a 1.5 T or 3.0 T commercially available system using an eight-channel breast array coil. The imaging sequences included a triplane localizing sequence followed by a sagittal fat-suppressed T₂-weighted sequence (e.g., repetition time/echo time (“TR/TE”), 4000-7000/85; section thickness, 3 mm; matrix, 256×192; field of view (“FOV”), 18-22 cm; no gap). A bilateral sagittal T₁-weighted fat-suppressed fast spoiled gradient-echo sequence (e.g., 17/2.4; flip angle, 35°; bandwidth, 31-25 Hz) was then performed before, and three times after, a rapid bolus injection (e.g., gadobenate dimeglumine/Multihance; Bracco Imaging, Princeton, N.J.; 0.1 mmol/kg) delivered through an IV catheter. Image acquisition started after contrast material injection and was obtained consecutively with each acquisition time of 120 seconds. Section thickness was 2-3 mm using a matrix of 256×192 and an FOV of 18-22 cm. Frequency was in the anteroposterior direction. After the examination, post-processing was performed including subtraction of the unenhanced images from the first contrast-enhanced images on a pixel-by-pixel basis and reformation of sagittal images to axial images.

### Exemplary Oncotype Dx RS

Each tumor specimen was transmitted to Genomic Health as standard of care and the Oncotype Dx RS was determined ranging from 0-100. Patients were classified into three groups based on the risk of recurrence 10 years after treatment: (i) low risk (group 1, RS <18), (ii) intermediate risk (group 2, RS 18-30), and (iii) high risk (group 3, RS >30).

### Computer-Based Image Analysis

Exemplary Image Preprocessing.

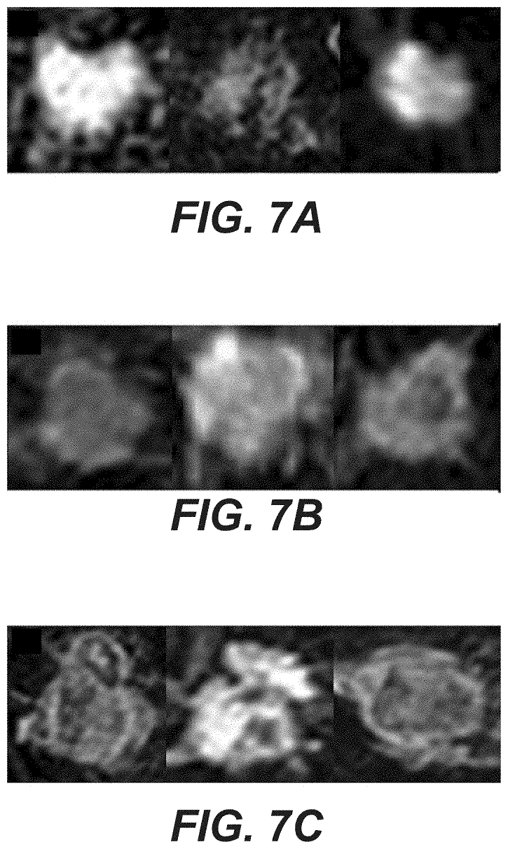

For all patients, breast tumor regions were manually annotated by a board-certified radiologist using a region-of-interest (“ROI”) drawn in 3DSlicer (see, e.g., Reference 46), based on first post-contrast DCE-MRI images. For 134 tumors, 1649 volumetric slices (e.g., mean 12.3 slices per tumor) in 32×32 voxel resolution were evaluated from the segmented tumor data. The intensity values at each pixel of the image were normalized by subtracting the mean intensity value of the image and dividing by the SD for each image. FIGS. 7A-7C show various views of a representative preprocessed single slice image of DCE-MRI breast tumors. For example, FIG. 7A is an exemplary set of DCE tumor images corresponding to a low Oncotype DX, FIG. 7B is an exemplary set of DCE tumor images corresponding to an intermediate Oncotype DX recurrence score, and FIG. 7C is an exemplary set of DCE tumor images corresponding to a high Oncotype DX recurrence score.

Exemplary Neural Network Architecture.

The exemplary CNN can be structured as a sequential set of convolution filters applied to the original image, followed by activation functions. The exemplary filters can apply learnable functions that can be trained with each new batch of input images. The filter weights can be updated by minimizing the cost function, which can compare the predicted output with ground truth training labels (e.g., an Oncotype Dx group). The L2 regularization, which can add a “squared magnitude” of a coefficient as a penalty term to the loss function, was used to discourage parameters of this learnable filter from becoming too large, and to prevent overfitting of the model to the training data. In the exemplary network, L2-norm (e.g., least squares error (“LSE”) was used on the fully connected layer. The exemplary L2-norm can minimize the sum of the square of the differences (S) between the target value (Yi) and the estimated values (f(xi), resulting in, for example:

$$S = {\sum }_{i = 1}^{n} {( {y}_{i} - f ( {x}_{i} ) )}^{2}$$

The exemplary activation function following convolutional filtering can introduce nonlinearities that can create a hierarchy of layers. This exemplary layered hierarchy can be used to facilitate depth in a network. Hierarchical depth in the network can facilitate filters to represent more complex features. The optimization of the network can include proper scaling of the input data and the learning rate step size. A proper preprocessing normalization of the data can be used to facilitate network convergence.

FIG. 8 illustrates an exemplary diagram of a further exemplary CNN according to an exemplary embodiment of the present disclosure. The exemplary CNN can be implemented using a series of 3×3 convolutional kernels to prevent overfitting. (See, e.g., Reference 48). Max-pooling with a kernel of 2×2 can be used. All non-linear functions can be modeled by the ReLU. (See, e.g., Reference 49). In deeper layers, the number of feature channels was increased from 32 to 64, reflecting increasing representational complexity. Dropout at 50% was applied to the second to last fully connected layer to prevent overfitting by limiting coadaptation of parameters. (See, e.g., Reference 50). Training was performed on over 200 epochs using the Adam optimizer with a base and a learning rate of 0.001. For better generalization and to prevent/reduce an overfitting of the model, a L2-regularization penalty of 0.01 was used.

As shown in the exemplary diagram of FIG. 8, a portion 810 of an image 805 can be input into the exemplary CNN. Image portion 810 can be input into a plurality of combined convolution and ReLu layers 815 (e.g., ten combined convolutional and ReLu layers). One or more maxpooling layers 820 can be located in between the combined convolution and ReLu layers 815. A dropout layer 825 can be located after the combined convolution and ReLu layers 815 and the maxpooling layers 820, which can feed into a one or more combined fully connected and ReLu layers 830. A softmax score 835 can be generated, which can be used to determine the breast cancer response.

As one example, for each breast tumor, a final softmax score threshold of 0.5 was used for classification. The softmax score, also known as softmax function, is a normalized exponential function. It can be a generalization of the logistic function that “squashes” a K-dimensional vector of arbitrary real values to a K-dimensional vector of real values, where each entry can be in the range (0, 1), and all the entries add up to 1. The softmax score provides the probability for each class label. The probability of each class can sum to 1 as dictated by the normalization constraint.

Two sets of experiments were performed, one three-class model to train the exemplary CNN model to predict low, moderate, or high Oncotype Dx RS and the second to predict two-class low vs. (e.g., moderate+high) Oncotype Dx RS. Five-fold cross-validation was performed with 80% of the data used as training and 20% used for testing purposes. In the three-class model, three different sensitivity and specificity metrics are provided, one for each class. The performance metrics can be calculated from the test dataset reserved for performance characterization upon which the training model was never exposed to. Training was implemented using the Adam optimizer, a procedure for first-order gradient-based optimization of stochastic objective functions, based on adaptive estimates of lower-order moments. (See, e.g., References 51 and 52). Parameters were initialized using a suitable heuristic. (See, e.g., Reference 53). To account for training dynamics, the learning rate can be annealed whenever training loss plateaus.

### Exemplary Statistical Analysis

An exemplary statistical analysis was performed. Age was calculated at the time of diagnosis. Descriptive statistics were used to summarize clinical, imaging, and pathologic parameters. Classification performance was evaluated using a multiclass receiver operating characteristics (“ROC”) analysis. This included generating ROC plots for each group versus the other two combined groups. For each of these two-class classifications, the sensitivity and specificity was reported.

### Exemplary Results

The tumor grade was 17.9% low grade (24/134), 65.7% intermediate grade (88/134), and 16.4% high grade (22-134). Axillary lymph node status was 92.5% negative (124/134) and 7.5% positive (10/134). Based on the American Joint Committee on Cancer, TNM classifications were as follows: T1 (73.8%, 99/134), T2 (25.4%, 34/134), T3 (0.7%, 1/134), T4 (0%); NO (92.5%, 124/134), N1 (7.5%, 10/134), N2 (0%), N3 (0%); M0 (100%, 134/134), M1 (0%). Most (97%, 130/134) of the patients had unifocal disease. Four patients had multifocal disease. Three out of four patients had one additional tumor. One out of four patients had two additional tumors. No contralateral tumors were present. Only the primary tumor that underwent Oncotype Dx evaluation was matched for MRI image analysis. The additional tumors did not undergo Oncotype Dx evaluation. Breast MRI was performed on 1.5 T in 61.2% (82/134) of the patients and on 3.0 T in 38.8% (52/134) of the patients. 11.2% (15/134) of the tumors demonstrated non-mass enhancement. 88.8% (119/134) of the tumors demonstrated mass enhancement.

The median Oncotype Dx score was 16 (range, 1-75). Patients were classified into three groups based on the risk of recurrence 10 years after treatment: low risk (group 1, RS <18), intermediate risk (group 2, RS of 18-30), and high risk (group 3, RS >30). The low-risk group consisted of 77 patients. The intermediate-risk group consisted of 40 patients. The high-risk group consisted of 17 patients.

A total of 134 breast cancer cases with Oncotype Dx recurrence scores were included. For each breast tumor, a final softmax score threshold of 0.5 was used for classification. The exemplary CNN was trained for a total of 200 epochs (e.g., batch size of 32) before convergence. Based on this, mean 5-fold validation accuracy was calculated. Initially, a three-class prediction model was utilized, classifying results into a low-risk group, intermediate-risk group, and high-risk group. The exemplary CNN achieved an overall accuracy of 81% (e.g., 95% confidence interval [CI]+4%). Subsequently, a two-class Oncotype Dx prediction model was evaluated in two groups consisting of 77 and 57 patients (e.g., group 1 vs. groups 2 and 3). The exemplary CNN achieved an overall accuracy of 84% (95% CI±5%) in two-class prediction.

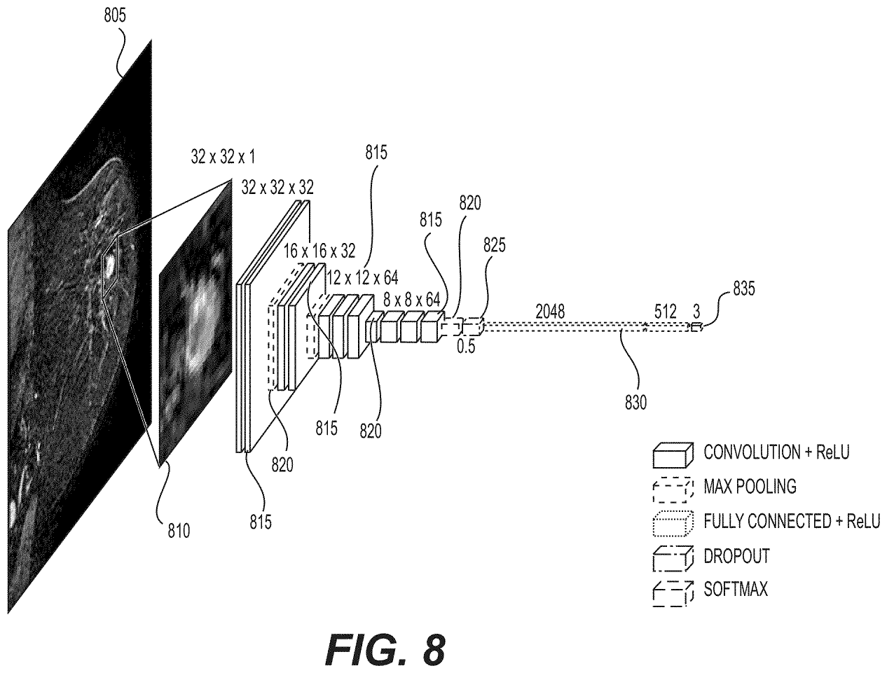

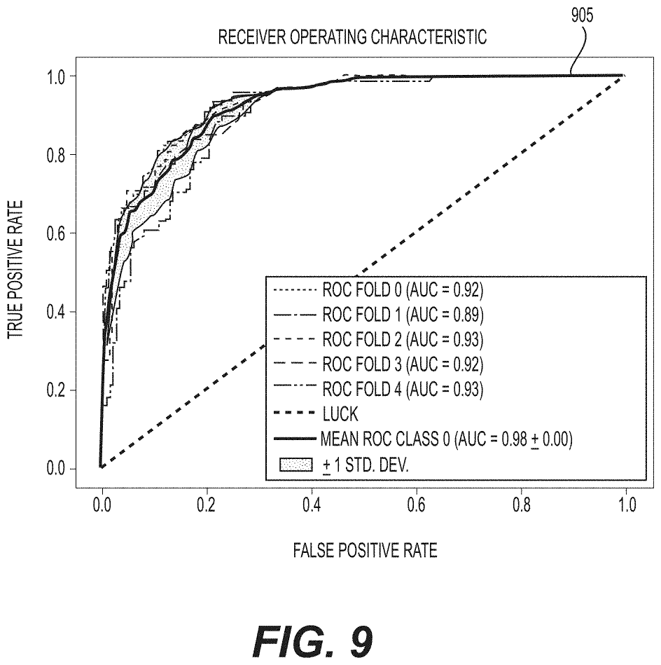

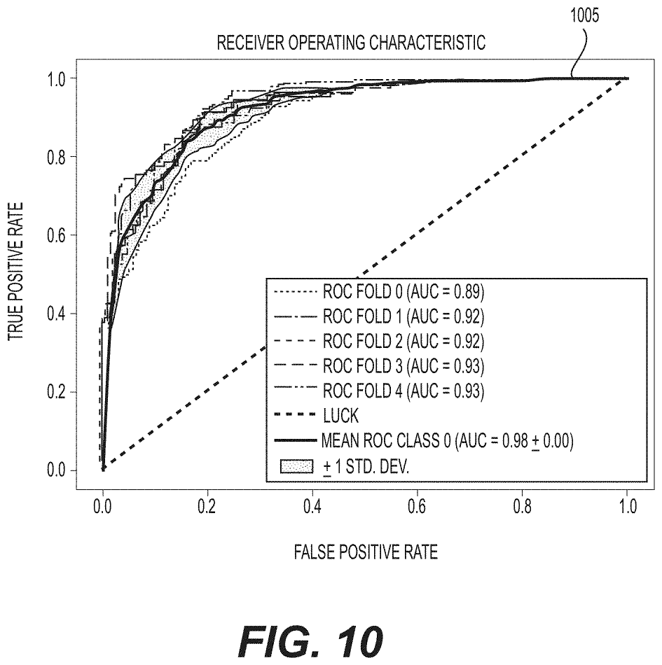

The exemplary ROC plot is shown in the graphs of FIGS. 9 and 10. For the exemplary three-class prediction model, the area under the ROC curve 905 was 0.92 (SD, 0.01) with specificity 90% (95% CI±5%) and sensitivity 60% (95% CI±6%). For the exemplary two-class prediction model, the area under the ROC curve 1005 was 0.92 (SD, 0.01) with specificity 81% (95% CI±4%) and sensitivity 87% (95% CI±5%).

The exemplary CNN achieved an overall accuracy of 84% in predicting patents with low Oncotype Dx RS compared to patients with intermediate/high Oncotype Dx RS. The exemplary results indicate the likelihood of utilizing the CNN procedure to predict Oncotype Dx RS.

### Exemplary Predicting Post Neoadjuvant Axillary Response

An exemplary analysis was performed 127 locally advanced breast cancer patients who: (i) underwent breast MRI before the initiation of NAC, (ii) successfully completed Adriamycin/Taxane-based NAC, and (iii) underwent surgery, including sentinel lymph node evaluation/axillary lymph node dissection with available final surgical pathology data. Data on tumor pathologic characteristics were obtained from the original pathology reports of the core biopsy specimen. Breast tumor receptors were determined based on IHC staining of the ER and PR interpreted according to the American Society of Clinical Oncology and College of American Pathologists Guidelines. Tumors were considered receptor positive if either ER or PR demonstrated ≥1% positive staining. (See, e.g., Reference 73). Tumors were considered HER2-positive if they were 3+ by immunohistochemistry or demonstrated gene amplification with a ratio of HER2/CEP17≥2 by in situ hybridization. (See, e.g., Reference 81).

Clinical and pathologic staging was determined based on the American Joint Committee on Cancer TNM Staging Manual, 7th edition. All patients included have biopsy-proven lymph node metastasis before NAC. After NAC, patients were classified into two groups based on their NAC response confirmed on final surgical pathology: pCR of the axilla (group 1), and non-pCR of the axilla (group 2).

### Exemplary Image Preprocessing

Images from all cases were normalized for signal intensity. An exemplary normalization of an image included, for example, subtracting the mean and dividing by the standard deviation for each image. Mean and standard deviation of gray levels were calculated across all data and applied pixel-wise to each individual image. To limit overfitting, data augmentation was performed in the form of translation, rotation, scaling, and shear of the original images was applied to aid in the training of a spatially invariant model.

### Exemplary Data Allocation and Image Segmentation

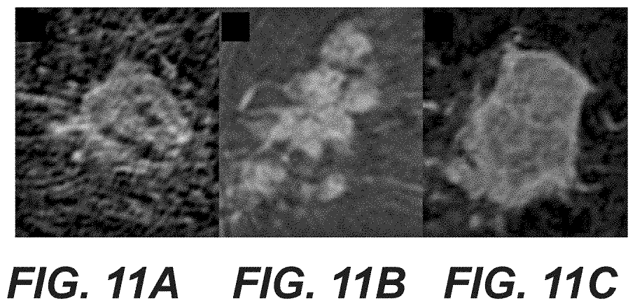

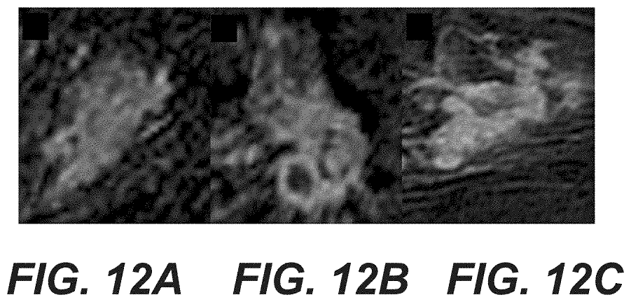

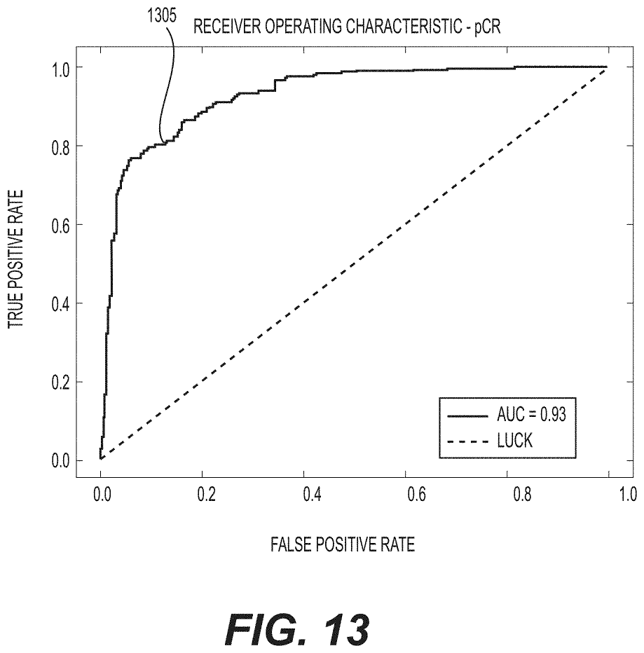

The cases were randomly separated into a training set, which included 80% of the cases, and a test set, which included 20% of the cases. The training data set was split into five class balanced folds for cross validated training. For each breast MRI, a tumor was identified on first set of T1 post-contrast dynamic images and underwent 3D segmentation using an open source software platform 3D Slicer. A total of 2811 slices from the 127 tumors were extracted with a threshold of 75 voxels per slice. From each slice that contained segmented tumor data, a patch of 64×64 pixels was extracted that completely contained the segmented tumor and was used for analysis. FIGS. 11A-11C show exemplary T1 post-contrast breast MRI images of tumors from patient with pCR of the axilla according to an exemplary embodiment of the present disclosure. FIGS. 12A-12C illustrate exemplary T1 post-contrast breast MRI images of tumors from patient with non-pCR of the axilla according to an exemplary embodiment of the present disclosure.

### Exemplary CNN Architecture

The exemplary system, method, and computer-accessible medium, according to an exemplary embodiment of the present disclosure, can utilize the exemplary CNN shown in FIG. 4 in order to predict post neoadjuvant axillary response.

### Exemplary CNN Training

The exemplary CNN was optimized with nadam (see, e.g., Reference 76), an adaptive moment estimation optimizer that utilizes nesterov momentum. The exemplary CNN was independently trained using k-fold cross validation. For each breast tumor, the maximum SoftMax score calculated by the exemplary CNN was used to predict pathologic response of the axilla. Code was implemented in open source software Keras with TensorFlow on a Linux workstation with NVIDIA GTX 1070 Pascal GPU.

### Exemplary CNN Testing

The trained CNN was used to predict classes on the earlier withheld testing dataset. Overall diagnostic performance in the form of sensitivity, specificity, and accuracy was reported with 95% confidence intervals. ROC curves are plotted as a function of different threshold criteria, as well as area under the ROC curve (“AUC”).

### Exemplary Results

Table 5 below indicates patient demographics and tumor characteristics. Patient population median age was 50 (range 23-82) years. The most frequent histologic tumor type was invasive ductal carcinoma 86.6% (100/127). The median size of the tumor was 3.2 (range 0.9-9.5) cm. Most of the tumor was either intermediate or high grade (96%, 122/127). Lymphovascular invasion was present in 33.9% (43/127) of the cases. Receptor status of tumors was: ER+, HER2−, 59 (46.5%), ER+, HER2+, 21 (16.5%), ER−, HER2+, 14 (11%), and ER−, HER2−, 33 (26%).

On final surgical pathology, 49 patients (38.6%, 49/127) achieved pCR of the axilla and 78 patients (61.4%, 78/127) did not with residual metastasis detected. Table 5 shows patient demographics and tumor characteristics stratified by the pCR of axilla and non-pCR of the axilla. Two class neoadjuvant prediction model of the axilla was evaluated for the two patient groups. Group 1 included of 49 patients with pCR of the axilla. Group 2 included of 78 patients with non-pCR of the axilla.

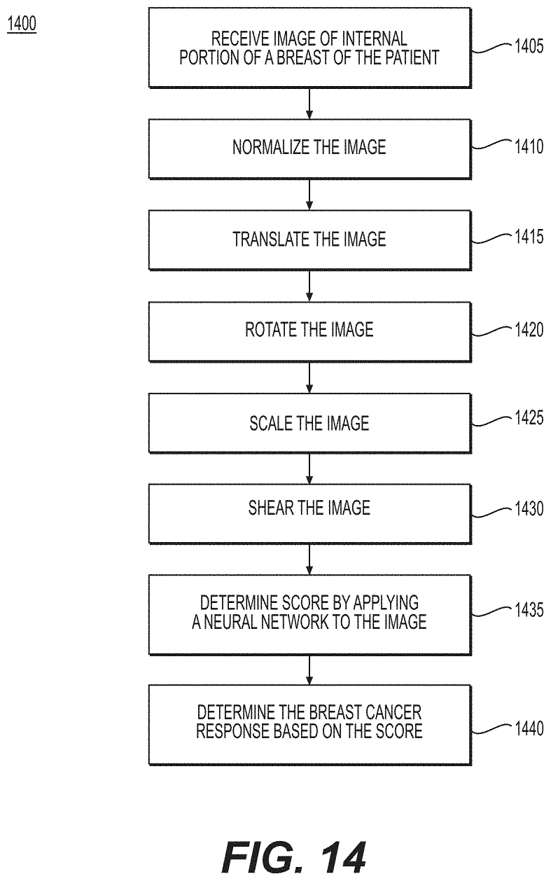

After 3D segmentation, a total of 2811 slices (e.g., min 2, max 41, median 18, and average 22) from the 127 tumors were extracted. A class-balanced separation of data allocated 80% for training and 20% for testing. The training data was split into five class balanced folds and independently trained five times. The following results report the average value over 95% confidence intervals of diagnostic performance of the model against training data. A final SoftMax score threshold of 0.5 was used for classification. The exemplary CNN achieved an overall accuracy of 83% (95% CI±5) with sensitivity of 93% (95% CI±6) and specificity of 77% (95% CI±4). FIG. 14 shows a graph of an exemplary ROC curve 1305 (0.93, 95% CI±0.04) according to an exemplary embodiment of the present disclosure.

Early prediction of axillary treatment response is beneficial in the management of locally advanced breast cancer with the potential to avoid the morbidity of ALND and create novel NAC combinations in non-responders. The nodal pCR rate was 38.5%, comparable to the 41.1% ACOSOG Z1071 overall axillary pCR rate. (See, e.g., Reference 78). Before the initiation of therapy, the exemplary CNN procedure achieved an overall accuracy of 83% in predicting NAC response in patients with node-positive breast cancer. Thus, the exemplary system, method, and computer-accessible medium can significantly improve on currently available prediction models, which depend on clinicopathologic information and post-NAC imaging analysis.

TABLE 5
Patients demographics and tumor characteristics of the entire
population and stratified by axillary pCR and axillary non-pCR

| | All | | Axillary pCR | | Axillary non-pCR | |
|---|---|---|---|---|---|---|
| Variable | (n = 127) | | (n = 49) | | (n = 78) | |
| Age at diagnosis, year, | 50 | (23-82) | 49 | (23-67) | 51 | (27-82) |
| median (range) | | | | | | |

Tumor histologic type, n (%)

| | | | | | | |
|---|---|---|---|---|---|---|
| Invasive ductal carcinoma | 68 | (53.5) | 27 | (55.1) | 41 | (52.6) |
| Invasive ductal carcinoma | 42 | (33.1) | 19 | (38.8) | 23 | (29.5) |
| and ductal carcinoma in situ | | | | | | |
| Invasive lobular carcinoma | 10 | (7.9) | 2 | (4.1) | 8 | (10.3) |
| Mixed ductal and lobular | 7 | (5.5) | 1 | (2.0) | 6 | (7.7) |
| carcinoma | | | | | | |
| Tumor size, cm, | 3.2 | (0.9-9.5) | 3.0 | (0.9-8.5) | 3.4 | (0.9-9.5) |
| median (range) | | | | | | |

Tumor grade, n (%)

| | | | | | | |
|---|---|---|---|---|---|---|
| Low | 5 | (3.9) | 0 | (0) | 5 | (6.4) |
| Intermediate | 44 | (34.6) | 15 | (30.6) | 29 | (37.2) |
| High | 78 | (61.4) | 34 | (69.4) | 44 | (56.4) |

Lymphovascular invasion

| | | | | | | |
|---|---|---|---|---|---|---|
| Present, n (%) | 43 | (33.9) | 18 | (36.7) | 25 | (32.1) |

Receptor status, n (%)

| | | | | | | |
|---|---|---|---|---|---|---|
| ER+, HER2− | 59 | (46.5) | 13 | (26.5) | 46 | (59) |
| ER+, HER2+ | 21 | (16.5) | 14 | (28.6) | 7 | (9.0) |
| ER−, HER2+ | 14 | (11.0) | 11 | (22.4) | 3 | (3.8) |
| ER−, HER2− | 33 | (26.0) | 11 | (22.4) | 22 | (28.2) |

ER estrogen receptor;
HER2 human epidermal growth factor 2

Overfitting can be an intrinsic limitation to CNN when using a relatively small dataset. In order to overcome this issue, over-fitting was minimized by application of suitable methods including, but not limited to, 50% dropout, data augmentation, and L2 regularization. Lastly, CNN is a type of artificial neural network, most recently developed due to advances in computer hardware technology. In contrast to traditional procedures, which utilize handcrafted tumor features based on human extracted patterns, neural networks facilitate the computer to automatically construct predictive statistical models, tailored to solve a specific problem subset. The laborious task of human engineers inputting specific patterns to be recognized can be replaced by inputting curated data and facilitating the technology to self-optimize and discriminate through increasingly complex layers. (See, e.g., Reference 72). Because training a CNN can be an end-to-end process, it does not clearly reveal the reasoning behind the final result in a deterministic manner. This can be an ongoing area of research to improve human understanding and intuition behind the predictions of a neural network.

The exemplary system, method, and computer-accessible medium, according to an exemplary embodiment of the present disclosure, can utilize an exemplary CNN to accurately predict axillary treatment response in node positive breast cancer using a baseline MRI tumor dataset. In non-responders, the exemplary system, method, and computer-accessible medium according to an exemplary embodiment of the present disclosure can impact clinical management to direct individualized treatment, minimize toxicity from ineffective agents, and explore novel neoadjuvant therapies. The exemplary CNN can further impact management of NAC responders, with the potential to avoid the morbidity of ALND and even SLNB.

FIG. 14 shows an exemplary flow diagram of a method for determining breast cancer response for a patient according to an exemplary embodiment of the present disclosure. For example, at procedure 1405, an image of an internal portion of a breast of the patient can be received. At procedure 1410, the image can be normalized. At procedure 1415, the image can be translated, at procedure 1420, the image can be rotated, at procedure 1425, the image can be scaled, and at procedure 1430, the image can be sheared. At procedure 1435, a score can be determined by applying a neural network to the image. At procedure 1440, the breast cancer response can be determined based on the score.

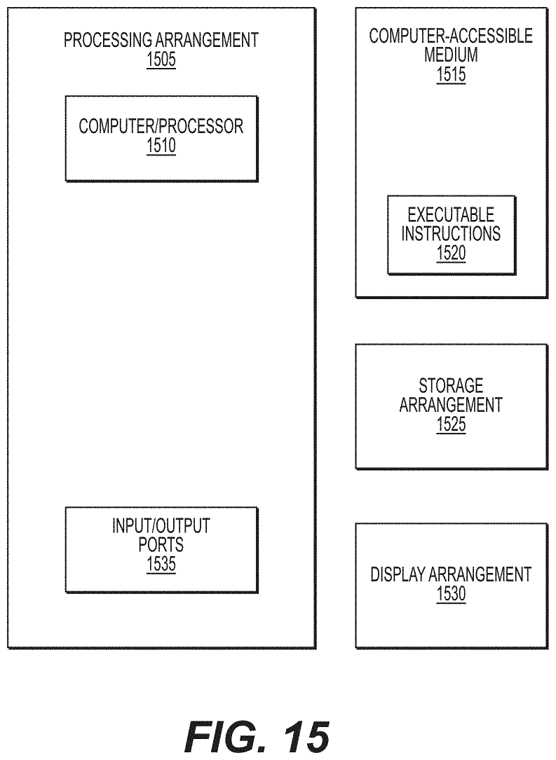

FIG. 15 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 1505. Such processing/computing arrangement 1505 can be, for example entirely or a part of, or include, but not limited to, a computer/processor 1510 that can include, for example one or more microprocessors, and use instructions stored on a computer-accessible medium (e.g., RAM, ROM, hard drive, or other storage device).

As shown in FIG. 15, for example a computer-accessible medium 1515 (e.g., as described herein above, a storage device such as a hard disk, floppy disk, memory stick, CD-ROM, RAM, ROM, etc., or a collection thereof) can be provided (e.g., in communication with the processing arrangement 1505). The computer-accessible medium 1515 can contain executable instructions 1520 thereon. In addition or alternatively, a storage arrangement 1525 can be provided separately from the computer-accessible medium 1515, which can provide the instructions to the processing arrangement 1505 so as to configure the processing arrangement to execute certain exemplary procedures, processes, and methods, as described herein above, for example.

Further, the exemplary processing arrangement 1505 can be provided with or include an input/output ports 1535, which can include, for example a wired network, a wireless network, the internet, an intranet, a data collection probe, a sensor, etc. As shown in FIG. 15, the exemplary processing arrangement 1505 can be in communication with an exemplary display arrangement 1530, which, according to certain exemplary embodiments of the present disclosure, can be a touch-screen configured for inputting information to the processing arrangement in addition to outputting information from the processing arrangement, for example. Further, the exemplary display arrangement 1530 and/or a storage arrangement 1525 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties:
1. Rastogi P, Anderson S J, Bear H D, et al: Preoperative chemotherapy: updates of National Surgical Adjuvant Breast and Bowel Project Protocols B-18 and B-27. J Clin Oncol 26:778-85, 2008.
2. Gianni L, Pienkowski T, Im Y H, et al: 5-year analysis of neoadjuvant pertuzumab and trastuzumab in patients with locally advanced, inflammatory, or early-stage HER2-positive breast cancer (NeoSphere): a multicentre, open-label, phase 2 randomised trial. Lancet Oncol 17:791-800, 2016.
3. Cortazar P, Zhang L, Untch M, et al: Pathological complete response and long-term clinical benefit in breast cancer: the CTNeoBC pooled analysis. Lancet 384:164-72, 2014.
4 Esserman L J, Berry D A, DeMichele A, et al: Pathologic complete response predicts recurrence-free survival more effectively by cancer subset: results from the I-SPY 1 TRIAL—CALGB 150007/150012, ACRIN 6657. J Clin Oncol 30:3242-9, 2012.
5. von Minckwitz G, Untch M, Blohmer J U, et al: Definition and impact of pathologic complete response on prognosis after neoadjuvant chemotherapy in various intrinsic breast cancer subtypes. J Clin Oncol 30:1796-804, 2012.
6. Wang-Lopez Q, Chalabi N, Abrial C, et al: Can pathologic complete response (pCR) be used as a surrogate marker of survival after neoadjuvant therapy for breast cancer? Crit Rev Oncol Hematol 95:88 104, 2015.
7. Broglio K R, Quintana M, Foster M, et al: Association of Pathologic Complete Response to Neoadjuvant Therapy in HER2-Positive Breast Cancer With Long-Term Outcomes: A Meta-Analysis. JAMA Oncol 2:751-60, 2016.
8. Carey L A: Neoadjuvant clinical trial designs: Challenges of the genomic era. Breast 24 Suppl 2:S88 90, 2015.
9. Carey L A, Winer E P: I-SPY 2—Toward More Rapid Progress in Breast Cancer Treatment. N Engl J Med 375:83-4, 2016.
10. Park J W, Liu M C, Yee D, et al: Adaptive Randomization of Neratinib in Early Breast Cancer. N Engl J Med 375:11-22, 2016.
11. Rugo H S, Olopade O I, DeMichele A, et al: Adaptive Randomization of Veliparib-Carboplatin Treatment in Breast Cancer. N Engl J Med 375:23-34, 2016.
12. von Minckwitz G, Blohmer J U, Costa S D, et al: Response-guided neoadjuvant chemotherapy for breast cancer. J Clin Oncol 31:3623-30, 2013.
13. Gu Y L, Pan S M, Ren J, et al: Role of Magnetic Resonance Imaging in Detection of Pathologic Complete Remission in Breast Cancer Patients Treated With Neoadjuvant Chemotherapy: A Meta-analysis. Clin Breast Cancer, 2017.
14. Li W, Arasu V, Newitt D C, et al: Effect of MR Imaging Contrast Thresholds on Prediction of Neoadjuvant Chemotherapy Response in Breast Cancer Subtypes: A Subgroup Analysis of the ACRIN 6657/I-SPY 1 TRIAL. Tomography 2:378-387, 2016.
15. Hylton N M, Blume J D, Bernreuter W K, et al: Locally advanced breast cancer: MR imaging for prediction of response to neoadjuvant chemotherapy—results from ACRIN 6657/I-SPY TRIAL. Radiology 263:663-72, 2012.
16. Hylton N M, Gatsonis C A, Rosen M A, et al: Neoadjuvant Chemotherapy for Breast Cancer: Functional Tumor Volume by MR Imaging Predicts Recurrence-free Survival-Results from the ACRIN 6657/CALGB 150007 I-SPY 1 TRIAL. Radiology 279:44-55, 2016.
17. Weis J A, Miga M I, Yankeelov T E: Three-dimensional Image-based Mechanical Modeling for Predicting the Response of Breast Cancer to Neoadjuvant Therapy. Comput Methods Appl Mech Eng 314:494-512, 2017.
18. Yankeelov T E: Integrating Imaging Data into Predictive Biomathematical and Biophysical Models of Cancer. ISRN Biomath 2012, 2012.
19. LeCun Y, Bengio Y, Hinton G: Deep learning. Nature 521:436-44, 2015.
20. Ravichandran K, Braman N, Janowczyk A, Madabushi A: A Deep Learning Classifier for Prediction of Pathological Complete Response to Neoadjuvant Chemotherapy from Baseline Breast DCE-MRI. SPIE Medical Imaging 2018: Computer-Aided Diagnosis 105750C, 2018.
21. Hammond M E, Hayes D F, Dowsett M, et al: American Society of Clinical Oncology/College Of American Pathologists guideline recommendations for immunohistochemical testing of estrogen and progesterone receptors in breast cancer. J Clin Oncol 28:2784-95, 2010.
22. Wolff A C, Hammond M E, Hicks D G, et al: Recommendations for human epidermal growth factor receptor 2 testing in breast cancer: American Society of Clinical Oncology/College of American Pathologists clinical practice guideline update. Arch Pathol Lab Med 138:241-56, 2014.
23. Pieper S, Halle M, Kikinis R. 3D Slicer. In: 2004 2nd IEEE International Symposium on Biomedical Imaging: Macro to Nano (IEEE Cat No 04EX821). IEEE; p. 632-5.
24. Drop out: Srivastava N, Hinton G E, Krizhevsky A, Sutskever I, Salakhutdinov R. Dropout: A Simple Way to Prevent Neural Networks from Overfitting. J Mach Learn Res. 2014; 15:1929-58.
25. Kingma D P, Ba J. Adam: A Method for Stochastic Optimization. arXiv:1412.6980
26. Ioffe, Sergey, and Christian Szegedy. “Batch normalization: Accelerating deep network training by reducing internal covariate shift.” International Conference on Machine Learning. 2015.
27. Therasse P, Arbuck S G, Eisenhauer E A, et al: New Guidelines to Evaluate the Response to Treatment in Solid Tumors. J Natl Cancer Inst 92:205-216, 2000
28. Li X, Arlinghaus L R, Ayers G D, et al: DCE-MRI analysis methods for predicting the response of breast cancer to neoadjuvant chemotherapy: pilot study findings. Magn Reson Med 71:1592-602, 2014
29. Li X, Abramson R G, Arlinghaus L R, et al: Multiparametric magnetic resonance imaging for predicting pathological response after the first cycle of neoadjuvant chemotherapy in breast cancer. Invest Radiol 50:195-204, 2015
30. Ah-See M L, Makris A, Taylor N J, et al: Early changes in functional dynamic magnetic resonance imaging predict for pathologic response to neoadjuvant chemotherapy in primary breast cancer. Clin Cancer Res 14:6580-9, 2008
31. Atuegwu N C, Arlinghaus L R, Li X, et al: Parameterizing the Logistic Model of Tumor Growth by DW-MRI and DCE-MRI Data to Predict Treatment Response and Changes in Breast Cancer Cellularity during Neoadjuvant Chemotherapy. Transl Oncol 6:256-64, 2013
32. Weis J A, Miga M I, Arlinghaus L R, et al: Predicting the Response of Breast Cancer to Neoadjuvant Therapy Using a Mechanically Coupled Reaction-Diffusion Model. Cancer Res 75:4697-707, 2015
33. Houssami N, Macaskill P, von Minckwitz G, et al. Meta-analysis of the association of breast cancer subtype and pathologic complete response to neoadjuvant chemotherapy. Eur J Cancer 48:3342-3352, 2012
34. Sorlie T, Perou C M, Tibshirani R, et al. Gene expression patterns of breast carcinomas distinguish tumor subclasses with clinical implications. Proc Natl Acad Sci USA 2001; 98:10869-10874
35. Carey L A, Perou C M, Livasy C A, et al: Race, breast cancer subtypes, and survival in the Carolina Breast Cancer Study. JAMA 295:2492-2502, 2006
36. Nguyen P L, Taghian A G, Katz M S, et al. Breast cancer subtype approximated by estrogen receptor, progesterone receptor, and HER-2 is associated with local and distant recurrence after breast-conserving therapy. J Clin Oncol. 2008 May 10; 26(14):2373-8.
37. Siegel R L. Cancer statistics, 2017. CA Cancer J Clin 2017; 67:7-30.
38. Perou C M, Sorlie T, Eisen M B, et al. Molecular portraits of human breast tumors. Nature 2000; 406:747-752.
39. Harris L N, Ismaila N, McShane L M, et al. Use of biomarkers to guide decisions on adjuvant systemic therapy for women with early-stage invasive breast cancer: American Society of Clinical Oncology Clinical Practice Guideline. J Clin Oncol 2016; 34:1134-1150.
40. Senkus, E, Kyriakides S, Ohno S, et al. Primary breast cancer: ESMO Clinical Practice Guidelines for diagnosis, treatment and follow-up. Ann Oncol 2015; 26(Suppl 5):v8-v30.
41. Paik S, Shak S, Tang G, et al. A multigene assay to predict recurrence of tamoxifen-treated, node negative breast cancer. N Engl J Med 2004; 351:2817-2826.
42. Paik S, Tang G, Shak S, et al. Gene expression and benefit of chemotherapy in women with node-negative, estrogen receptor-positive breast cancer. J Clin Oncol 2006; 24:3726-3734.
43. Gluz O, Nitz U A, Christgen M, et al. West German Study Group Phase III PlanB Trial: First prospective outcome data for the 21-gene recurrence score assay and concordance of prognostic markers by central and local pathology assessment. J Clin Oncol 2016; 34:2341-2349.
44. Song J L, Chen C, Yuan J P, et al. Progress in the clinical detection of heterogeneity in breast ca45. Fan M, Li H, Wang S, et al. Radiomic analysis reveals DCE-MRI features for prediction of molecular subtypes of breast cancer. PloS one 2017; 12: e0171683.
46. LeChun Y, Bengio T, Hinton G. Deep learning. Nature 2015; 521: 436-444.
47. Pieper S, Halle M, Kikinis R. 3D Slicer. In: 2004 2nd IEEE International Symposium on Biomedical Imaging: Macro to Nano (IEEE Cat No 04EX821). IEEE 2004; 632-635.
48. Simonyan K, Zisserman A. Very deep convolutional networks for large-scale image recognition. International Conference on Learning Representations 2015; p 1-14.
49. Nair V, Hinton G E. Rectified linear units improve restricted boltzmann machines. In Proceedings of the 27th international conference on machine learning (ICML-10) 2010; pp. 807-814.
50. Srivastava N, Hinton G E, Krizhevsky A, et al. Dropout?: A simple way to prevent neural networks from overfitting. J Mach Learn Res 2014; 15: 1929-1958.
51. Kingma D P, Ba J. Adam: A method for stochastic optimization. 2014; arXiv preprint arXiv:1412.6980.
52. Mandic D P. A generalized normalized gradient descent algorithm. IEEE Signal Process Lett 2004; 11:115-118.
53. He K, Zhang X, Ren S, et al. Delving deep into rectifiers: Surpassing human-level performance on ImageNet classification. arXiv:1502.01852 (2015).
54. Abadi M, Barham P, Chen J, et al. TensorFlow: A system for large-scale machine learning. In: 12th USENIX Symposium on Operating Systems Design and Implementation (OSDI '16) [Internet] 2016; p 265-84.
55. Orucevic A, Heidel R E, Bell J L. Utilization and impact of 21-gene recurrence score assay for breast cancer in clinical practice across the Unites States: Lessons learned from the 2010 to 2012 National Cancer Data Base analysis. Breast Cancer Res Treat 2016; 157:427-435.
56. Morrow M, Waters J, Morris E. MRI for breast cancer screening, diagnosis, and treatment. Lancet 2011; 378:1804-1811.
57. Ashraf A B, Daye D, Gavenonis S, et al. Identification of intrinsic imaging phenotypes of breast cancer tumors: Preliminary associations with gene expression profiles. Radiology 2014; 272:374-384.
58. Sutton E J, Oh J H, Dashevsky B Z, et al. Breast cancer subtype intertumor heterogeneity: MRI-based features predict results of a genomic assay. J Magn Reson Imaging 2015; 42:1398-1406.
59. Li H, Zhu Y, Burnside E S, et al. MR imaging radiomics signatures for predicting the risk of breast cancer recurrence as given by research versions of MammaPrint, Oncotype D X, and PAM50 gene assays. Radiology 2016; 281:382-391.
60. Sun C, Shrivastaval A, Singh S, et al. Revisiting Unreasonable Effectiveness of Data in Deep Learning Era. arXiv preprint arXiv:1707.02968 (2017).
61. Teshome M, Hunt K K. Neoadjuvant therapy in the treatment of breast cancer. Surg Oncol Clin N Am. 2014; 23(3):505-23.
62. Hunt K K, Yi M, Mittendorf E A, et al. Sentinel lymph node surgery after neoadjuvant chemotherapy is accurate and reduces the need for axillary dissection in breast cancer patients. Ann Surg. 2009; 250:558-66.
63. Rastogi P, Anderson S J, Bear H D, et al. Preoperative chemotherapy: updates of National Surgical Adjuvant Breast and Bowel Project Protocols B-18 and B-27. J Clin Oncol. 2008; 26:778-85.
64. Akay C L, Meric-Bernstam F, Hunt K K, et al. Evaluation of the M D Anderson Prognostic Index for local-regional recurrence after breast conserving therapy in patients receiving neoadjuvant chemotherapy. Ann Surg Oncol. 2012; 19:901-7.
65. Mamounas E P, Anderson S J, Dignam J J, et al. Predictors of locoregional recurrence after neoadjuvant chemotherapy: results from combined analysis of National Surgical Adjuvant Breast and Bowel Project B-18 and B-27. J Clin Oncol. 2012; 30(32):3960-6.
66. Cortazar P, Zhang L, Untch M, et al. Pathological complete response and long-term clinical benefit in breast cancer: The CTNeoBC pooled analysis. Lancet. 2014; 384:164-72.
67. von Minckwitz G, Untch M, Blohmer J-U, et al. Definition and impact of pathologic complete response on prognosis after neoadjuvant chemotherapy in various intrinsic breast cancer subtypes. J Clin Oncol. 2012; 30:1796-804.
68. Wang-Lopez Q, Chalabi N, Abrial C, et al. Can pathologic complete response (pCR) be used as a surrogate marker of survival after neoadjuvant therapy for breast cancer? Crit Rev Oncol Hematol. 2015; 95:88-104.
69. Hennessy B T, Hortobagyi G N, Rouzier R, et al. Outcome after pathologic complete eradication of cytologically proven breast cancer axillary node metastases following primary chemotherapy. J Clin Oncol. 2005; 23:9304-11.
70. Lobbes M, Prevos R, Smidt M, et al. The role of magnetic resonance imaging in assessing residual disease and pathologic complete response in breast cancer patients receiving neoadjuvant chemotherapy: a systematic review. Insights Imaging 2013; 2:163-75.
71. Weber J J, Jochelson M S, Eaton A, et al. MRI and prediction of pathologic complete response in the breast and axilla after neoadjuvant chemotherapy for breast cancer. J Am Coll Surg. 2017; 225(6):740-6.
72. LeCun Y, Bengio Y, Hinton G. Deep learning. Nature. 2015; 521:436-44.
73. Hammond M E, Hayes D F, Dowsett M, et al. American Society of Clinical Oncology/College Of American Pathologists guideline recommendations for immunohistochemical testing of estrogen and progesterone receptors in breast cancer. J Clin Oncol. 28:2784-95, 2010.
74. Simonyan K, Zisserman A. Very deep convolutional networks for large-scale image recognition. In: ICLR, 2015.
75. Srivastava N, Hinton G, Krizhevsky A, et al. Dropout: a simple way to prevent neural networks from overfitting. J Mach Learn Res. 2014; 15:1929-58.
76. Kingma D P, Ba J A. A method for Stochastic Optimization. arXiv:1412.6980 [cs.LG], December 2014.
77. Ioffe S, Szegedy C. Batch normalization: accelerating deep network training by reducing internal covariate shift. In: International Conference on Machine Learning. 2015.
78. Boughey J C, Suman V J, Mittendorf E A, et al. Sentinel lymph node surgery after neoadjuvant chemotherapy in patients with node-positive breast cancer: the ACOSOG Z1071 (Alliance) Clinical Trial. JAMA. 2013; 310:1455-61.
79. Mattingly A E, Mooney B, Lin H Y, et al. Magnetic resonance imaging for axillary breast cancer metastasis in the neoadjuvant setting: a prospective study. Clin Breast Cancer. 2017; 17(3):180-7.
80. Harlow S P, Krag D N, Julian T B, et al. Prerandomization Surgical Training for the National Surgical Adjuvant Breast and Bowel Project (NSABP) B-32 trial: a randomized phase III clinical trial to compare sentinel node resection to conventional axillary dissection in clinically node-negative breast cancer. Ann Surg. 2005; 241(1):48-54.
81. Tan V K, Goh B K, Fook-Chong S, et al. The feasibility and accuracy of sentinel lymph node biopsy in clinically node-negative patients after neoadjuvant chemotherapy for breast cancer a systematic review and meta-analysis. JSurg Oncol. 2011; 104:97-103.

1. A non-transitory computer-accessible medium having stored thereon computer-executable instructions for determining at least one breast cancer response for at least one patient, wherein, when a computer arrangement executes the instructions, the computer arrangement is configured to perform procedures comprising:
receiving at least one image of at least one internal portion of a breast of the at least one patient; and
determining the at least one breast cancer response by applying at least one neural network to the at least one image.

2. The computer-accessible medium of claim 1, wherein the at least one breast cancer response is a response to at least one chemotherapy treatment.

3. The computer-accessible medium of claim 1, wherein the at least one breast cancer response includes an Oncotype DX recurrence score.

4. The computer-accessible medium of claim 1, wherein the at least one breast cancer response is a neoadjuvant axillary response.

5. The computer-accessible medium of claim 1, wherein the at least one image is at least one magnetic resonance image (MRI).

6. The computer-accessible medium of claim 5, wherein the at least one MRI includes at least one dynamic contrast enhanced MRI.

7. The computer-accessible medium of claim 1, wherein the neural network includes a convolutional neural network (CNN).

8. The computer-accessible medium of claim 7, wherein the CNN includes a plurality of layers.

9. The computer-accessible medium of claim 8, wherein the layers include (i) a plurality of combined convolutional and rectified linear unit (ReLu) layers, (ii) a plurality of max pooling layers, (iii) at least one combined fully connected and ReLu layer, and (iv) at least one dropout layer.

10. The computer-accessible medium of claim 9, wherein (i) the combined convolutional and rectified linear unit (ReLu) layers include at least ten combined convolutional and rectified linear unit (ReLu) layers, and (ii) the max pooling layers include at least four max pooling layers.

11. The computer-accessible medium of claim 10, wherein (i) two of the at least ten combined convolutional and rectified linear unit (ReLu) layers have 64×64×64 feature channels, (ii) two of the at least ten combined convolutional and rectified linear unit (ReLu) layers have 32×32×128 feature channels, (iii) three of the at least ten combined convolutional and rectified linear unit (ReLu) layers have 16×16×128 feature channels, and (iv) three of the at least ten combined convolutional and rectified linear unit (ReLu) layers have 8×8×512 feature channels.

12. The computer-accessible medium of claim 1, wherein the computer arrangement is further configured to determine at least one score based on the at least one image using the at least one neural network.

13. The computer-accessible medium of claim 12, wherein the computer arrangement is configured to determine the at least one breast cancer response based on the score.

14. The computer-accessible medium of claim 13, wherein the computer arrangement is configured to determine the at least one breast cancer response based on the score being above 0.5.

15. The computer-accessible medium of claim 1, wherein the computer arrangement is further configured to normalize the at least one image.

16. The computer-accessible medium of claim 15, wherein the computer arrangement is configured to normalize the at least one image by subtracting a mean for a plurality of images of further internal portions of further breasts, and dividing by a standard deviation for the at least one image.

17. The computer-accessible medium of claim 1, wherein the computer arrangement is further configured to (i) translate the at least one image, (ii) rotate the at least one image, (iii) scale the at least one image, and (iv) shear the at least one image.

18. The computer-accessible medium of claim 1, wherein the computer arrangement is further configured segment the at least one image prior to applying the at least one neural network.

19. A method for determining at least one breast cancer response for at least one patient, comprising:
receiving at least one image of at least one internal portion of a breast of the at least one patient; and
using a computer arrangement, determining the at least one breast cancer response by applying at least one neural network to the at least one image.

20-36. (canceled)

37. A system for determining at least one breast cancer response for at least one patient, comprising:
a computer hardware arrangement configured to:
receive at least one image of at least one internal portion of a breast of the at least one patient; and
determine the at least one breast cancer response by applying at least one neural network to the at least one image.

38-54. (canceled)
